SEQUENCE LISTING (23 sequences)

SEQ ID NO: 1 - protein, 154 aa, Artificial Sequence
  Description of Artificial Sequence  A POLYPEPTIDE COMMON TO GCM PROTEINS OF VARIOUS SPECIES
WDINDXXXPXXXXXXDXFXXWXXXXXXXIYSXXXXXAXXHXSXWAMRNTNNHNXXILKKSCLGVXXCXXXCXXXXGXXXXLRPAICDKARXKQQXKXCPXXNCXXXLXXXXCRGHXGXPVTXFWRXDGXXIXFQXKGXHDXPXPEXKXXXEXRR

SEQ ID NO: 2 - DNA, 8 bp, Artificial Sequence
  Description of Artificial Sequence A DNA SEQUENCE TO WHICH A GCM PROTEIN COMPRISING THE AMINO ACID OF SEQ ID NO 1 BINDS
rcccgcat

SEQ ID NO: 3 - DNA, 20 bp, Artificial Sequence
  Description of Artificial Sequence SYNTHETIC DNA
tgtgtggaattgtgagcgga

SEQ ID NO: 4 - DNA, 19 bp, Artificial Sequence
  Description of Artificial Sequence SYNTHETIC DNA
ggttttcccagtcacgacg

SEQ ID NO: 5 - DNA, 54 bp, Artificial Sequence
  Description of Artificial Sequence SYNTHETIC DNA
tgtgtggaattgtgagcggacctacccgcattacgggttttcccagtcacgacg

SEQ ID NO: 6 - DNA, 54 bp, Artificial Sequence
  Description of Artificial Sequence SYNTHETIC DNA
tgtgtggaattgtgagcggatatactaatttgttaggttttcccagtcacgacg

SEQ ID NO: 7 - DNA, 14 bp, Artificial Sequence
  Description of Artificial Sequence DNA FRAGMENT
aaatcccgcataaa

SEQ ID NO: 8 - DNA, 14 bp, Artificial Sequence
  Description of Artificial Sequence DNA FRAGMENT
catgcccgcgtcgc

SEQ ID NO: 9 - DNA, 14 bp, Artificial Sequence
  Description of Artificial Sequence DNA FRAGMENT
atggcgcgcatcca

SEQ ID NO: 10 - DNA, 14 bp, Artificial Sequence
  Description of Artificial Sequence DNA FRAGMENT
catacccgcagttt

SEQ ID NO: 11 - DNA, 14 bp, Artificial Sequence
  Description of Artificial Sequence DNA FRAGMENT
cagacccacataat

SEQ ID NO: 12 - DNA, 14 bp, Artificial Sequence
  Description of Artificial Sequence DNA FRAGMENT
tttgcccgcatttc

SEQ ID NO: 13 - DNA, 14 bp, Artificial Sequence
  Description of Artificial Sequence DNA FRAGMENT
ccggcccgcattga

SEQ ID NO: 14 - DNA, 14 bp, Artificial Sequence
  Description of Artificial Sequence DNA FRAGMENT
aatgcccacatgat

SEQ ID NO: 15 - DNA, 14 bp, Artificial Sequence
  Description of Artificial Sequence DNA FRAGMENT
agtaccagcatctg

SEQ ID NO: 16 - DNA, 14 bp, Artificial Sequence
  Description of Artificial Sequence DNA FRAGMENT
accaccctcatgag

SEQ ID NO: 17 - DNA, 14 bp, Artificial Sequence
  Description of Artificial Sequence DNA FRAGMENT
cgcgccagcatgaa

SEQ ID NO: 18 - protein, 436 aa, Homo sapiens
MEPDDSDSEDKEILSWDINDVKLPQNVKKTDWFQEWPDSYAKHIYSSEDKNAQRHLSSWAMRNTNNHNSRILKKSCLGVVVCGRDCLAEEGRKIYLRPAICDKARQKQQRKRCPNCDGPLKLIPCRGHGGFPVTNFWRHDGRFIFFQSKGEHDHPKPETKLEAEARRAMKKVNTAPSSVSLSLKGSTETRSLPGETQSQGSLPLTWSFQEGVQLPGSYSGHLIANTPQQNSLNDCFSFSKSYGLGGITDLTDQTSTVDPMKLYEKRKLSSSRTYSSGDLLPPSASGVYSDHGDLQAWSKNAALGRNHLADNCYSNYPFPLTSWPCSFSPSQNSSEPFYQQLPLEPPAAKTGCPPLWPNPAGNLYEEKVHVDFNSYVQSPAYHSPQGDPFLFTYASHPHQQYSLPSKSSKWDFEEEMTYLGLDHCNNDMLLNLCPLR

SEQ ID NO: 19 - protein, 436 aa, Mus musculus
MELDDFDPEDKEILSWDINDVKLPQNVKTTDWFQEWPDSYVKHIYSSDDRNAQRHLSSWAMRNTNNHNSRILKKSCLGVVVCSRDCSTEEGRKIYLRPAICDKARQKQQRKSCPNCNGPLKLIPCRGHGGFPVTNFWRHDGRFIFFQSKGEHDHPRPETKLEAEARRAMKKVHMASASNSLRMKGRPAAKALPAEIPSQGSLPLTWSFQESVQLPGTYSTPLIANAPQQKSLNDCLSFPKNYDLGGSTELEDPTSTLDSMKFYERCKFSSSRIYGSEEQFQPPVPGTYGDYEDLQTWNKNVALGRNPSDDIYYPAYPLPVASWPYDYFPSQNSLEHLPQQVPSEPPAAQPGCHPLWSNPGGEPYEEKVSVDLSSYVPSLTYHPPQQDPFLLTYGSPTQQQHALPGKSNRWDFEEEMACMGLDHFNNEMLLNFCSLR

SEQ ID NO: 20 - protein, 504 aa, Mus musculus
MPADSTQDEDAVLSYGMKLTWDINDPQMPQEPTHFDHFREWPDGYVRFIYSSQEKKAQRHLSGWAMRNTNNHNGHILKKSCLGVVVCARACALKDGSHLQLRPAICDKARLKQQKKACPNCHSPLELVPCRGHSGYPVTNFWRLDGNAIFFQAKGVHDRPRPESKSETEGRRSALKRQMASFYQPQKRRSEEPEARSTQDIRGHLNSTAALEPTELFDMTADTSFPIPGQPSPSFPNSDVHRVTCDLPTFQGDIILPFQKYPNPSIYFPGPPWGYELASSGVTGSSPYSTLYKDSSVVPDDPDWIPLNSLQYNVSSYGSYERTLDFTARYHSWKPTHGKPSLEEKVDCEQCQAVPTSPYYNLELPCRYLPVPAAGTQALQTVITTTVAYQAYQHPALKHSDSMQEVSSLASCTYASENLPMPIYPPALDPQEGVIRAASPSGRAPLKVPGDCQAPRPTLDFPQEADPSGTDGADVWDVCLSGVGSVMGYLDRTGQPFSFDDEDF

SEQ ID NO: 21 - protein, 504 aa, Drosophila
MVLNGMPITMPVPMPVPMPVPSPPATKSRVAIDWDINDSKMPSVGEFDDFNDWSNGHCRLIYSVQSDEARKHASGWAMRNTNNHNVNILKKSCLGVLLCSAKCKLPNGASVHLRPAICDKARRKQQGKCCPNRNCNGRLEIQACRGHCGYPVTHFWRRDGNGIYFQAKGTHDHPRPEAKGSTEARRLLAGGRRVRSLAVMLARESALSDKLSSLRPTKRQAKTQSIQESKRRRMGASDVLETKQELVVPPTTYLPTSTPTHSTNFNQSQGSYVPAGQGSVISQWNREIHYETEDPCYANGMYSYDMLHSPLSAHSSTGSYYQENKPQQLQHSQYQQQLSPQQHVPVSYDPSQPISSSLQCGMPSYEICDDTSSLTSSSGYCSEDYGYYNGYLPNSLDVSNGSQSQNLSQDASIYTTSSEIFSVFESTLNGGGTSGVDLIYDEATAYQQHQQQGTFPHLTNYQQEPQDQMQSADYYYSNTGVDSNWNIQMDATYHPVNSTDPIYC

SEQ ID NO: 22 - protein, 35 aa, Mus musculus
ILKKSCLGVVVCGRDCLAEEGRKIYLRPAICDKAR

SEQ ID NO: 23 - protein, 47 aa, Drosophila
WAMRNTNNHNVNILKKSCLGVLVCSQHCTLPNGSKINLRPAICDKAR

(19) United States
(12) Patent Application Publication
(10) Pub. No.: US 2002/0081718 A1
(43) Pub. Date: Jun. 27, 2002

(54) POLYPEPTIDE, AND A DNA SEQUENCE TO WHICH A PROTEIN CONTAINING THE POLYPETIDE BINDS

(76) Inventor: YOSHIKI HOTTA, SHIZUOKA (JP)

Correspondence Address: WENDEROTH LIND & PONACK, 2033 "K" STREET N.W., SUITE 800, WASHINGTON, DC, 20006

(21) Appl. No.: 08/980,068

(22) Filed: Nov. 26, 1997

(30) Foreign Application Priority Data
Nov. 28, 1996 (JP) 318285/1996

(51) Int. Cl. C07H 21/04; C12N 15/00; H01L 21/338; C12N 15/09; C12N 15/63; C12N 15/70; C12N 15/74; C07K 1/00; C07K 14/00; C07K 17/00

(52) U.S. Cl. 435/320.1; 536/23.5; 530/350

(57) ABSTRACT

The present invention provides a polypeptide existing in common to proteins expressed from the glial cells missing (gcm) gene which is a fate determination gene of cells in animal central nervous system, which polypeptide has the amino acid sequence of Sequence ID No. 1 or a part thereof and a DNA sequence to which the proteins containing the polypeptide binds. These polypeptide and DNA sequence would contribute to the progress of clarification of the forming mechanism of the human nervous systems, and at the same time, opens up a new way to development of diagnostic and medical treatment methods of cerebral functional diseases and cerebral tumor.

### FIELD OF THE INVENTION

The present invention relates to a polypeptide existing in common to proteins expressed from the glial cells missing (gcm) gene which is a fate determination gene of cells in animal central nervous system, and DNA sequence on animal gemonic DNA to which the polypeptide binds. More particularly, the present invention relates to a polypeptide existing in novel DNA-binding protein and a specific DNA sequence on the genomic DNA to which this protein binds, which are useful as materials for research purposes for searching for the mechanism of central nervous system formation in various animal species including human, and useful for technical development of diagnosis and medical treatment means of various diseases caused by structural and functional disorders of the central nervous system.

### BACKGROUND ART OF THE INVENTION

The central nervous system can be deemed as a giant information processor comprising a huge number of cells, and component cells may be broadly divided into neuron and glial cells. The neurons are considered to play a central role in information processing by forming a network, and glial cells provide support to formation and functioning of the network and plays an important role in repairing damages to the network. Accurate formation of these two types of cells playing different roles as described above is an essential prerequisite for the central nervous system to function normally. It is therefore very important, in clarifying the forming mechanism of the complicated nervous system and searching for functions thereof, to have knowledge of formation of diversity and determination of differentiation of generation of nervous system.

The fact that neurons and glial cells are produced from common precursor cells (stem cells) has so far been reported for many species. However, it has not as yet been clarified by what mechanism individual stem cells are differentiated into neurons and glial cells. While it is known that some molecules exert an effect on the determination of differentiation, it has not been shown that this effect is necessary for determination of glia vs. neuronal cell fate.

Drosophila is a model organism having a long history as an experimental material in genetics. The most important advantage of using Drosophila is the possibility of rapidly identifying an unknown gene participating in an interesting biological process through screening of mutants. It is now possible to determine individual cells of the central nervous system and know the generating process thereof in detail as a result of preparation of cell markers using various antibodies or lacZ gene and establishment of dye injecting techniques into a single cell. The central nervous system of Drosophila is accordingly becoming an excellent model for research efforts on generation of cells forming the nervous system and differentiation determination mechanism.

The central nervous system of Drosophila is composed of 30 neuroblasts per side of each segment. Neuroblasts produce both neurons and glial cells by repeating a stem cell-like asymmetric divisions, and about 300 neurons and about 30 glial cells are created per side of each segment before completion of a nervous system. Each of the 30 neuroblasts is formed at a predetermined timing at a predetermined position to permit individual identification, so that a name is assigned to each of them. Further followup of cells produced by specific neuroblasts permits observation of generation of specific neurons and glial cells. In Drosophila, therefore, fate of each cell of the nervous system is almost completely determined genetically.

The present inventors carried out screening of mutants of Drosophila for the purpose of identifying genes participating in differentiation of the nervous system, and as a result, the present inventors obtained a mutant strain having an abnormality in formation of glial cells. The resultant mutant was therefore named glial cells missing (gcm), and the gcm gene was specified as a causative gene of this mutant (Cell 82: 1025-1036, 1995). This mutation of the gcm gene, which confirms the fact that cells to become glia are differentiated into neurons, or that misexpressing gcm gene in neuroblasts causes presumptive neurons to differentiate into glia, suggests that this gcm gene controls determination of fate between neurons and glial cells.

Apart from the present inventors, three groups carried out analysis independently as to this gcm gene, and obtained results supporting the conclusion as described above (Cell 82: 1013-1023, 1995; Genetics 139: 1663-1678, 1995; Development 122: 131-139, 1996; Cell 83: 671-674, 1995; Neuron 15: 1219-1222, 1995).

As described above, it is now clear that the gcm gene plays an important role in the determination of fate of nervous system cells. However, amino acid sequence deduced from the nucleotide sequence of cDNA thereof exhibits almost no homology with any of the proteins in the database, so that functions of the protein (GCM) expressed from the gcm gene have been unknown.

In order to accurately understand functions of a particular gene, in general, it is essential to clarify the physiological activity of the expression product. It is therefore very important to specify the role of GCM in the central nervous system, with a view to elucidating the formation mechanism of the central nervous system as well as to develop diagnosis and medical treatment techniques of central nervous system diseases.

The present invention was made in view of the circumstances as described above, and has an object to provide a polypeptide and DNA sequence which permit more diverse and various industrial uses of the gcm gene and the expressed protein thereof, by clarifying functions of the expressed protein GCM of the gcm gene of nervous system cells.

### SUMMARY OF THE INVENTION

The present invention provides a polypeptide existing in common to proteins expressed from the gcm gene which is a fate determination gene of cells of animal central nervous system, which polypeptide has the amino acid sequence of sequence ID No. 1 or a part thereof.

The invention provides also a polypeptide, of which one or more amino acid residues in the Sequence ID No. 1 or a part thereof are substituted, deleted or added.

Further, the invention provides a DNA sequence of animal genomic DNA to which a protein containing the foregoing polypeptide binds, which DNA sequence has the nucleotide sequence of Sequence ID No. 2 or a homolog sequence thereto, and a vector holding such DNA sequence.

In the amino acid sequence of Sequence ID No 1, Xaa represents an arbitrary amino acid residue, and in the nucleotide sequence of Sequence ID No. 2, R represent A or G.

The polypeptide provided by the present invention and the DNA sequence to which a protein containing this polypeptide binds are useful as materials for research purposes of searching for the mechanism of the central nervous system formation in various animal species and functions thereof. These materials are applicable also for diagnosis and medical treatment of various diseases caused by structural or functional disturbances of the central nervous system (for example, glial cerebral tumor). In the future, it is expected to be possible to grow and culture blast glial cells, separated from a neonatal, in vitro, and when he or she, becoming adult, suffers from a cerebral functional disorder, to control the gcm genes in the stored cells by means of the foregoing protein or polypeptide, convert blast glial cells into neurons, return the thus converted neurons into the brain to recover functions: the “self-brain cell transplantation technique”.

### BRIEF DESCRIPTION OF THE DRAWINGS

FIG. 1A is a constitutional diagram of GCM fusion protein used in the gel shift assay; and

FIG. 1B is an electrophoresis showing the result of the gel shift assay;

FIG. 2A illustrates the matching ratio of the GCM binding sequence; and FIG. 2B is an electrophoresis showing the result of an antagonistic assay;

FIG. 3A illustrates GCM binding sites in the upstream region of repo gene; and

FIG. 3B is a DNA sequence in these sites;

FIG. 4 illustrates GCM amino acid sequences of human, mouse and Drosophila, respectively; and

FIG. 5 illustrates partial amino acid sequences of GCM from mouse and Drosophila.

### DETAILED DESCRIPTION OF THE INVENTION

The polypeptide of the invention can be isolated through hydrolysis or the like of GCM protein expressed in the central nervous system of an animal. It is also possible to prepare the polypeptide of the invention through chemical synthesis on the basis of the amino acid sequence provided by the invention. The polypeptide of the invention includes a peptide fragment containing any partial amino acid sequence of Sequence ID No. 1 (five or more amino acid residues).

The DNA sequence of the invention is a DNA sequence existing in common to genomic DNA sequences to which GCM protein binds, and can be prepared by synthesizing with a knows method as a DNA fragment. Such a DNA fragment is applicable as a probe for specifying a gene to which GCM protein binds, or a primer for PCR-amplifying such a gene.

Now, the polypeptide and the DNA sequence of the invention will be described in detail on the basis of an experimental study carried out by the present inventors.

(1) DNA binding properties of GCM

The gcm gene of Drosophila encodes a 504 amino acid protein. It is clear, after searching for amino acid sequences in the database, that this protein has no motif characterized to date, other than a nuclear localization signal. This protein GCM was consequently confirmed to be quite a novel nuclear protein. Because this protein brings about expression and other changes of many genes, it was most simply assumed that GCM directly binds to genomic DNA and serves as a transcription regulating factor controlling the expression of its target genes. With a view to studying this possibility, the DNA binding properties of GCM were investigated.

The upstream region of the repo gene of Drosophila was selected as an target of GCM binding. The homeobox gene repo is expressed in virtually all glial cells from an early stage onwards, and this expression is strongly dependent on gcm. Therefore, when assuming that GCM is a transcription regulating factor, repo gene is considered suitable as candidates for target genes.

a) Procedures

Fusion proteins of various regions of GCM and maltose-binding protein were prepared as an expression products of Escherichia coli, and binding or not with various DNA fragments prepared from the genomic upstream region of repo gene was investigated by the gel shift assay.

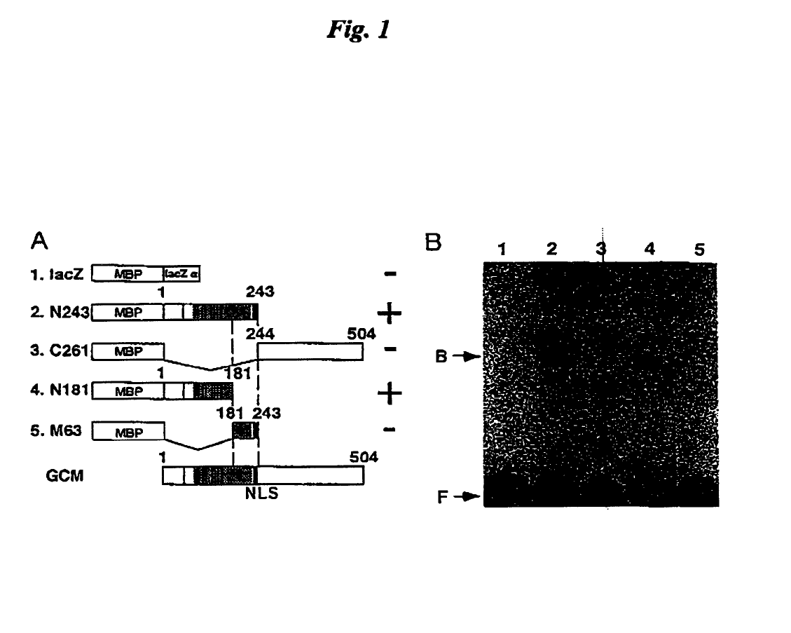

Production of fusion proteins: Fusion proteins were prepared using a protein and purification system (made by New England Biolabs). Various segments of the gcm gene were amplified by the PCR method with pfu polymerase (TOYOBO), and inserted in a pMAL™-cz vector, which was then introduced into Escherichia coli BL21(DE3)pLysS. Fusion proteins and MBP-lacZp, which was used as a control for gel shift assay, were induced in the transformed Escherichia coli, and then sonicated, extracted and affinity-purified using amylose resin. Structure of each fusion protein is as shown in FIG. 1A: 1 is the control; 2-5 are fusion proteins; and 6 is a schematic view of a normal GCM. Figures attached to each constitutional diagram represent positions of the amino acid residues of GCM.

Preparation of DNA fragments in repo gene upstream region: Clones containing the repo gone were isolated from a Drosophila genomic library. After confirming the nucleotide sequence thereof, the repo gene upstream region of about 7 kb was excised with multiple restriction enzymes to prepare 60-460 bp DNA fragments.

Gel shift assay: Each fusion protein in an amount of about 200 ng was incubated in accordance with a known method (Cell 64: 439-446, 1991; EMBO J. 10:2965-2973, 1991), with 100 bp of ³²P-labeled DNA at 25° C. for 30 minutes, and then subjected to electrophoresis at 4° C. with polyacrylamide.

b) Results

The results of the gel shift assay are shown, in the electrophoresis of FIG. 1B. The lane numbers correspond to proteins of which the structures are shown in FIG. 1A. The fusion proteins 2 (N243) and 4 (N181) bound to the DNA fragments in the repo gene upstream region (arrow B in the drawing). This permits confirmation that GCM is a DNA-binding protein and at the same time, suggests that DNA-binding activity is present in the region of up to the 181st amino acid at the amino terminal of GCM. Since the amino acid sequence of up to the 181st one does not exhibit homology with any known proteins, it was found to be a novel DNA-binding domain.

(2) DNA sequence to which GCM binds

It was investigated whether or not the DNA-binding domain of GCM binds by recognizing a specific sequence.

a) Procedures

Gel shift assay: In accordance with a known method (Science 250: 1104-1110, 1990; Cell 64: 459-470, 1991; Science 257: 1951-1955, 1992; Cell 68: 283-302, 1992), probes were prepared from oligonucleotide resulting from insertion of 15 bp of random sequence, respectively, into nucleotide sequences of Sequence ID Nos. 3 and 4, and gel shift assay was carried out in the same manner as above by causing reaction with fusion protein N243. Oligonucleotide binding with fusion protein was isolated, and PCR-amplified with the sequences of Sequence ID Nos. 3 and 4 as primers, and the resultant PCR products and fusion protein were examined with gel shift assay. This cycle was repeated three times, and the sequences of the finally obtained PCR products were determined.

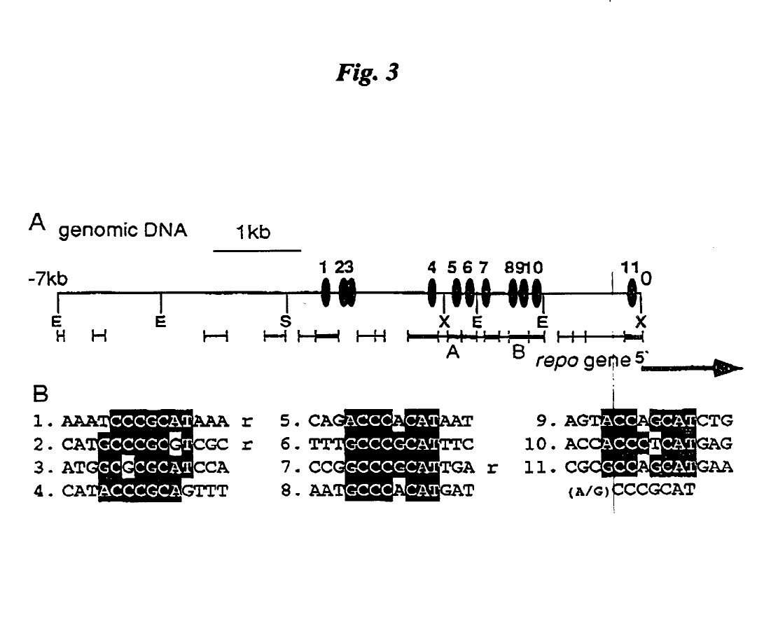

Competition assay: Competitive oligonucleotide exhibiting the nucleotide sequences of Sequence ID Nos. 5 and 6 were previously caused to react with fusion protein N243, and then with N243 with a labeled 200 bp DNA fragment (FIGS. 3A and 3B) to carry out a gel shift assay in the same manner as above.

b) Results

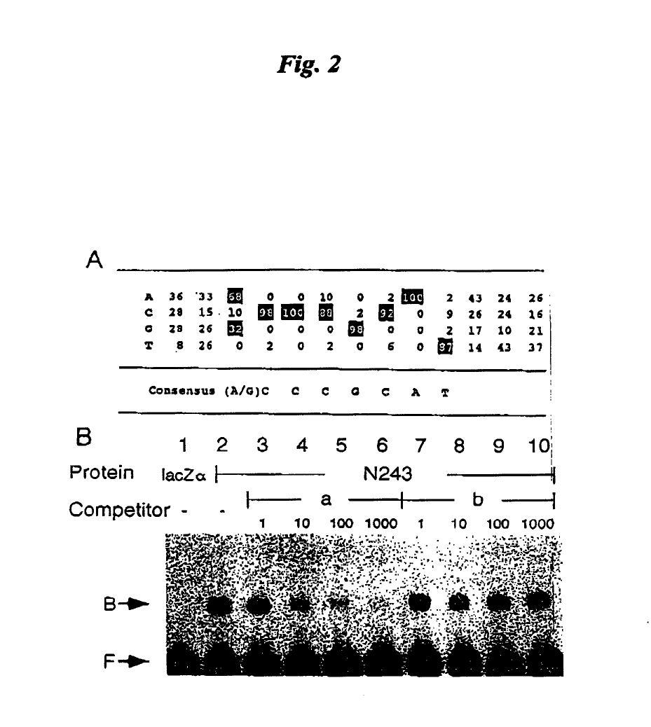

Forty-eight clones of oligonucleotide were obtained through the gel shift assay of oligonucleotide and fusion protein and repetition three times of PCR-amplification of oligonucleotide bound to protein. Among them, 39 clones had common nucleotide sequences shown by Sequence ID No. 2. Eleven clones had sequences that contained only one base mismatch, and the remaining three clones had only two mismatches of base. The sequence alignment of each base is as shown in FIG. 2A, which shows a high alignment within a range of from 87 to 100%. This result strongly suggests that GCM protein specifically recognizes the nucleotide sequence of Sequence ID No. 2 upon binding to DNA.

The Sequence ID No. 2 was confirmed to be a GCM-binding sequence also from the results of the competition assay (FIG. 2B). More specifically, when previously causing a reaction with the competitive oligonucleotide of Sequence ID No. 5 including the sequence of Sequence ID No. 2 (FIG. 2B-a), binding property with the probe disappears in dependence upon the concentration thereof (1, 10, 100 and 1,000 times), whereas in a case with Sequence ID No. 6 not including a sequence of Sequence ID No. 2, no competitive action against the probe was observed.

(3) GCM binding sites in the upstream region of the repo gene

On the assumption that the repo is a target gene of GCM, the region in the upstream of the repo gene for GCM binding sites was searched for.

Firstly, gel shift assays were carried out by the use of 21 non-overlapping DNA fragments (FIG. 3A: bottom horizontal bar) and the fusion protein N243 to specify the DNA fragments to which the protein binds.

As a result, N243 bound to eight DNA fragments (FIG. 3A: bottom thick horizontal bar). On the other hand, C261 did not bind to any of the DNA fragments.

Then, the DNA sequence of the 7 kb upstream region was investigated, and it was found that eleven GCM-binding sequences (Sequence ID No. 2) existed within 4 kb upstream region, while no binding sequence was present in the −4 to −7 kb upstream region. The sequence of these 11 sites are as shown in FIG. 3B: those having seven sequences in match out of eight bases were counted as GCM-binding sites. All the eight foregoing DNA fragments binding to N243 contained one or more GCM-binding sequences.

The fact that as many as eleven sites of GCM-binding sequence clusters are present in the upstream region of the repo gene suggests that GCM directly controls expression of the repo gene as a transcription regulating factor.

(4) GCM common sequence in different species of animal

In order to find whether the DNA binding domain is conserved throughout evolution, mammalian homologues were compared with Drosophila GCM.

A human gene (hGCMa) was derived from the EST database. Because the sequences in this database were not complete, a complete code sequence list was prepared by the 5′-RACE (rapid amplification of cDNA ends) and 3′-RACE methods. Mouse genes (nGCMa and mGCMb) were isolated by using the conserved region between GCM and hGCMa. More specifically, mGCMa was prepared from mouse placenta poly(A)+RNA by the reverse transcriptase PCR(RT-PCR) method, and mGCMb from mouse brain poly(A)+RNA by the RT-PCR method.

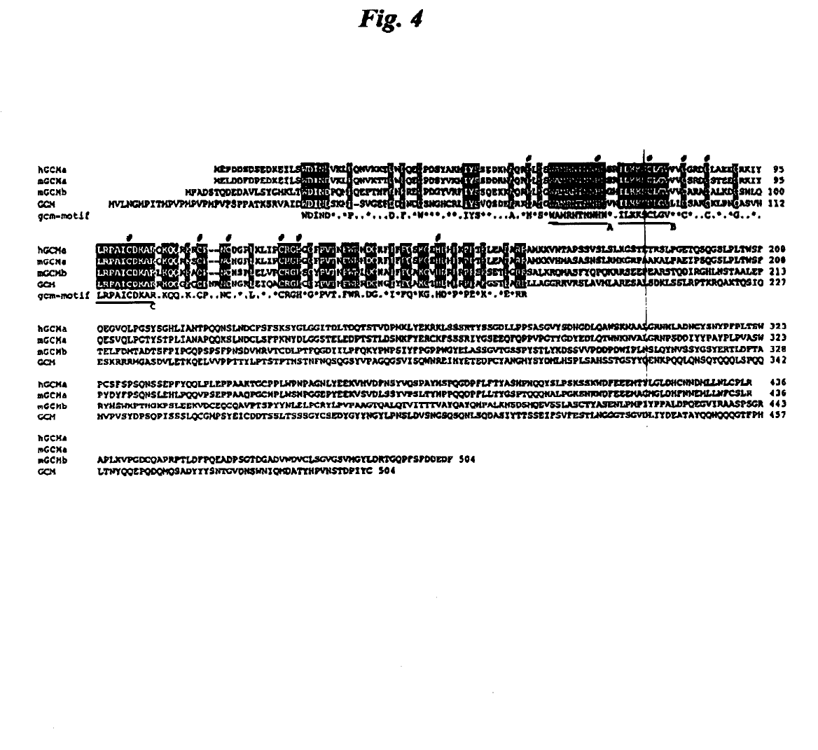

Comparison of the amino acid sequence of the protein deduced from these human and mouse genes and the amino acid sequence of GCM revealed strong conservation of the highly basic amino-terminal one-third as shown in FIG. 4. An evolutionally conserved motif could be unambiguously defined from these comparisons, the defined motif being named the “gcm-motif”. Further, this motif corresponds also to the DNA-binding domain of GCM (1-181 amino acid residues). Comparison of the individual sequences reveals the presence of three absolutely conserved stretches of nine to ten amino acid residues (A, B and C in FIG. 4), and seven conserved cysteine and four conserved histidine residues In contrast to the highly conserved gcm-motif at the amino-terminal regions, carboxy-terminal regions mostly have no similarity to each other nor to any known proteins.

Gel shift assays and competition assays were carried out in the same manner as above by using proteins obtained by causing expression of DNA fragments of up to the 171-st amino acid residue of hGCMa, giving results similar to those of GCM. These results also suggest that the gcm-motif is a sequence containing a specific DNA-binding domain.

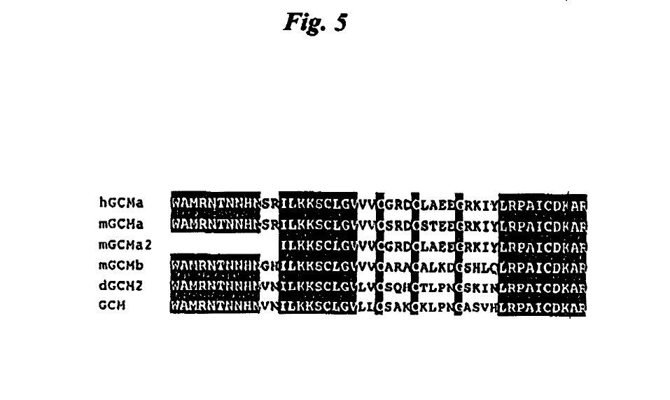

Further, presence of gcm-motif was confirmed also in the RT-PCR product (mGCMa2) resulting from the mouse brain poly (A)+RNA as a template and the PCR product (dGCM2) resulting from the Drosophila genomic DNA as template (FIG. 5) suggests that the gcm-motif is conserved commonly to many animal species, and the proteins containing this sequence form a novel family of DNA-binding proteins.

According to the present invention, as described above in detail, the glial cells missing (gcm) gene of the central nervous system of various animals commonly exist in expressed GCM proteins, and there are provided a polypeptide having a specific amino acid sequence functioning as a DNA-binding domain of this GCM protein, and a DNA sequence to which this GCM protein binds. This contributes to the progress of clarification of the molecular mechanism of forming the human nervous systems, and at the same time, opens up a new way to development of diagnostic and medical treatment methods of cerebral functional diseases and cerebral tumor using the gcm genes, expressed proteins thereof and peptide.

What is claimed is:

1. A polypeptide existing in common to proteins expressed from the glial cells missing (gcm) gene which is a fate determination gene of cells in animal central nervous system, which polypeptide has the amino acid sequence of Sequence ID No. 1 or a part thereof.

2. The polypeptide of claim 1, of which one or more amino acid residues are substituted, deleted or added.

3. A DNA sequence on animal genomic DNA to which a protein containing the polypeptide of claim 1 or 2 binds, which DNA sequence has the nucleotide sequence of Sequence ID No. 2 or a homologous sequence thereto.

4. A vector holding the DNA sequence of claim 3.
